(19) United States
(12) Patent Application Publication
(10) Pub. No.: US 2004/0039413 A1
(43) Pub. Date: Feb. 26, 2004

(54) Radial artery compression system

(75) Inventors: Dan Akerfeldt, Uppsala (SE); Krister Hornfeldt, Uppsala (SE)

Correspondence Address: FOLEY AND LARDNER, SUITE 500, 3000 K STREET NW, WASHINGTON, DC, 20007, US

(73) Assignee: RADI MEDICAL SYSTEMS AB

(21) Appl. No.: 10/224,586

(22) Filed: Aug. 21, 2002

(51) Int. Cl. A61B 17/00

(52) U.S. Cl. 606/201

(57) ABSTRACT

A splint (2) for a radial artery compression system (1) or an arterial wrist support, which is provided with one or more longitudinal reinforcement profiles (10a, 10b) on the bottom surface thereof. Whereby the reinforcement profiles (10a, 10b) form a support structure (11), such that the splint (2) when placed on a flat surface is supported in one position without wobbling. The splint (2) may further be comprised of a non-rigid material such as polystyrene, paper (preferably recycled) and the like, such that a disposable splint (2) may be achieved.

### FIELD OF THE INVENTION

The present invention relates to a radial artery compression system or an arterial wrist support. More specifically, the invention relates to a splint for a radial artery compression system or an arterial wrist support, wherein the wrist is securely fixated before compression of the artery, and which spread the compression pressure over a large area on the back of the hand, wrist and forearm, such that increased comfort is achieved, and which gives enhanced support during handling.

### BACKGROUND OF THE INVENTION

Following an invasive medical procedure such as catheterization or similar invasive medical procedure, the blood flow through the puncture wound has to be stopped, so that haemostasis may begin as fast as possible. This may be done using a radial artery occluder, comprising a splint and a pressure strap, such as the system proposed in U.S. Pat. No. 5,601,597 that is described in detail below. A similar splint is disclosed in U.S. Pat. No. 4,798,199. When performing catheterization and the like, it is of great importance that the wrist be kept firmly at a specified angle throughout the whole procedure.

U.S. Pat. No. 4,798,199 discloses an arterial wrist support for invasive monitoring of a patient's blood gases during and after surgery. The apparatus is designed to support a patient's hand, wrist, and at least a portion of the patient's forearm in a relaxed position which allows for efficient starting of the arterial line and continuous maintenance, observation and cleaning of the arterial site. The apparatus may also be effectively used in the administration of intravenous care.

More specifically, the arterial wrist support according to U.S. Pat. No. 4,798,199 includes a substantially rigid, lightweight, unitary molded body anatomically shaped to accommodate the contours of a patient's arm and to position the patient's wrist at the proper angle to expose the radial artery for accurate and efficient handling of the arterial puncture. Wide resilient straps adapted to be releasably secured to the molded body are provided to attach the body to the patient's arm and thus immobilize the patient's wrist. The molded body may also include a foam pad lining located on its upper surface and extending along its length to cushion the patient's hand and wrist and thus provide a soft comfortable support.

U.S. Pat. No. 5,601,597 discloses a combination radial artery occluder and wrist splint to prevent blood flow through a puncture wound or incision following catheterization or similar invasive medical procedure. This device generally includes three main components: a wrist splint that extends along the distal end of the patient's forearm and the back of the wrist and hand; an adjustable pressure strap attached to the splint that extends around the distal end of the forearm; and an adjustable securing strap also attached to the splint that extends around the palm of the patient's hand. The wrist splint is preferably generally trough-shaped so as to cradle the forearm, wrist, and hand around at least approximately half the circumference thereof. Also, the wrist splint preferably includes padding on its inner, concave surface to cushion the forearm. The pressure strap includes a pressure pad that is selectively positioned over the wound in the forearm or wrist to occlude blood flow through only the radial artery while allowing blood flow to the hand through the ulnar artery. During use, the adjustable pressure strap is slowly tightened over the wound until radial artery blood flow has stopped at the wound. This aids haemostasis in the wound in the radial artery but allows the ulnar artery to deliver enough blood to the hand to ensure tissue viability. In addition, the securing strap is tightened around the palm of the hand to help immobilize the wrist joint.

### SUMMARY OF THE INVENTION

In U.S. Pat. No. 5,601,597 the inventors claim that they solve the problem of delay in haemostasis caused by movement of the wounded tissue, such as wrist movement after a radial artery cardiac catheterization procedure. Tissue stretching and contraction caused by movement of a limb joint can prevent clotting and inadvertently reopen wounds. But as the pressure strap and the pressure pad are located at the forearm end of the splint, the pressure on the puncture wound varies if the patient tries to move the wrist in the splint, whereby the haemostasis can be delayed. Further, during compression of the radial artery, this device only spreads out the pressure from the pressure strap over a relatively small area on back of the forearm. Still further, this design is limited to be used for compressing wounds within a limited range along the extension of the artery, as the pressure strap is arranged in a fixed position on the splint.

Both the support of U.S. Pat. No. 4,798,199 and the splint of U.S. Pat. No. 5,601,597 are molded structures that preferably are comprised of a rigid or semi rigid plastic material such as PVC or the like, which preferably also have a soft padding on the side that will be in contact with the hand. Therefore, these products are expensive to produce and will have to be reused, which implies that they have to be sterilized every time they are to be used, which further increases the costs.

An improved radial artery compression system is needed, which provides increased patient comfort, and which, compared with existing devices, is cheap to produce such that a disposable radial artery compression system may be achieved.

An object of the invention is to provide a device that overcomes the drawbacks of the prior art devices. This is achieved by the radial artery compression system described below.

One advantage with such a radial artery compression system is increased patient comfort, due to a large contact area on the back of the hand/wrist/forearm, and light weight.

Another advantage is that the radial artery compression system described below is disposable, and is comprised of cheap, lightweight, and recyclable materials.

Still a further advantage is that the radial artery compression system described below may be formed to have a supporting structure, which stabilizes the device during use.

### BRIEF DESCRIPTION OF THE DRAWINGS

FIG. 1 is a top view in perspective of a radial artery compression system according to the invention.

FIG. 2 is a bottom view in perspective of a radial artery compression system according to the invention.

FIG. 3 is a front view in perspective of a radial artery compression system according to the invention.

FIG. 4 is a perspective view of another embodiment of the invention.

### DETAILED DESCRIPTION OF THE PREFERRED EMBODIMENTS

Embodiments of the invention will now be described with reference to the figures.

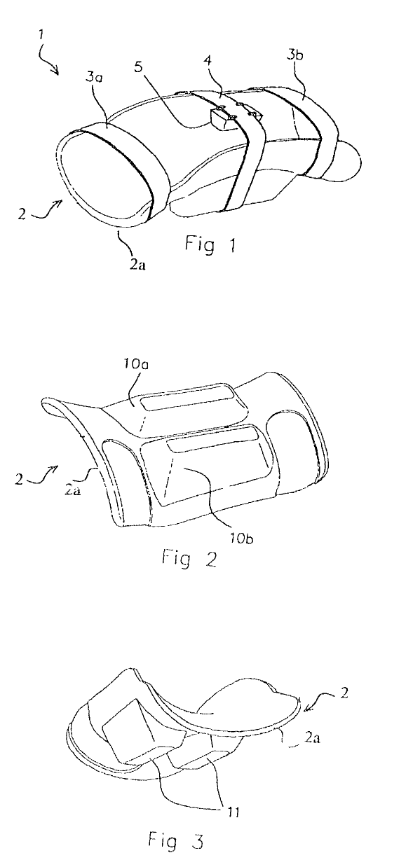

FIG. 1 shows a preferred embodiment of a radial artery compression system according to the invention. The system comprises a splint 2 having a main body 2a, a first and a second strap 3a, 3b, a compression strap 4, and a compression pad 5. The first and second straps 3a, 3b are preferably arranged at the longitudinal ends of the splint 2 and fixate the hand, wrist and forearm in the splint 2. When the hand, wrist and forearm are fixated in the splint 2, the compression strap 4 is used to put pressure on the compression pad 5, which is to be positioned over the wound in the forearm or wrist to occlude blood flow through only the radial artery while allowing blood flow to the hand through the ulnar artery.

The inner surface of the splint 2 is preferably generally trough-shaped so as to cradle the forearm, wrist and hand around approximately half the circumference thereof. As can be seen in FIG. 2, the outer surface of the splint 2 generally has the same shape as the top surface, apart from one or more essentially longitudinally arranged reinforcement profiles 10a, 10b. A reinforcement profile is a portion of the splint which has an area of increased thickness as compared to the rest of the splint. If the main body 2a of the splint has a thickness of T, the longitudinal reinforcement profile extends beyond the main body by an additional thickness of at least T, and preferably by at least 2T, and further preferably by at least 3T. In the embodiment shown in FIGS. 1 to 3, these profiles 10a, 10b extend along a fraction of the length of the splint 2. These reinforcement profiles 10a, 10b greatly increase the rigidity and the torsional rigidity of the splint 2, whereby the splint may be produced with reduced material thickness, or using cheap non-rigid materials such as polystyrene, paper (preferably recycled), and the like. In this embodiment, the main body 2a and the reinforcement profiles 10a, 10b are formed as one piece from the same material. By lowering the material consumption or by using such cheap materials a disposable radial artery compression system 1 is achieved.

The reinforcement profiles 10a, 10b may further form a support structure 11 for the splint 2, whereby the splint 2 when placed on a flat surface is firmly supported in one position without wobbling, which can be seen in FIG. 3. To form the support structure 11, the bottom surfaces of the reinforcement profiles 10a, 10b are arranged to lie in the same plane, below which plane no parts of the splint 2 may extend. This supporting feature facilitates over all handling when using the radial artery compression system 1 of the invention, especially when strapping the compression pad 5 to the wound. The supporting structure 11 also gives increased stability if the splint 2 is used as a support when performing invasive medical procedures such as catheterization, wherein accurate and efficient handling of the arterial puncture is of great importance.

The support structure 11 formed by the reinforcement profiles 10a, 10b may further be shaped such that the hand positioned in the splint 2 will be placed in a predefined angle with respect to the surface on which the support structure 11 of the splint 2 is placed. In an alternative embodiment the reinforcement profiles 10a, 10b may be formed so as to mate a non-flat surface or a matching fixture for fixation of the splint 2, e.g. a dovetail structure or the like.

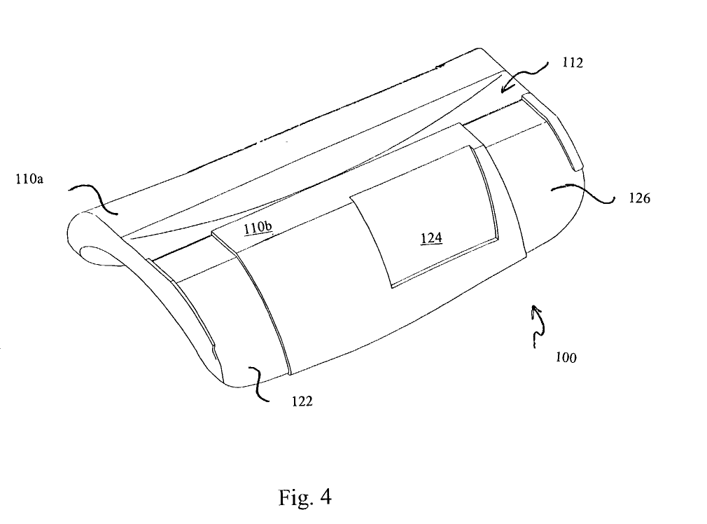

FIG. 4 is a bottom perspective view of another embodiment 100 of the invention. In this embodiment, one or more reinforcement profiles 110a, 110b extend along substantially the entire length of the splint. The bottom surface(s) of the reinforcement profile(s) lie in the same plane to allow the splint to rest on a surface without wobbling. In the FIG. 4 embodiment, two reinforcement profiles are shown, separated by a recess 112 (which saves material); however the recess may be omitted and essentially one large reinforcement profile may be provided. The top portion of the splint 100 is similar to that of splint 2. In FIG. 4, strap recesses 122, 124, 126 are provided for straps (not shown). Recesses may also be provided on portion 110a. The straps generally go around the patient and the splint, similar to that shown in FIG. 1. The straps may be attached to the splint and/or themselves using a hook and loop type fastener, self-adhesive, or other suitable technique. Even if a strap lies between the splint and the surface on which the splint is resting, because the support structure of the splint is comprised of flat surface(s), the splint will rest without wobbling. The recess 124 is sized to allow some longitudinal movement of the strap within recess 124. The entire contents of Swedish application 0101040-4, filed Mar. 23, 2001, is incorporated herein by reference.

The invention is not limited to the embodiments described above. Variations and modifications of the invention will occur to those skilled in this field after receiving the above teachings. The invention is, therefore, defined with reference to the following claims.

What is claimed is:

1. A splint for a radial artery compression system comprising at least one longitudinal reinforcement profile arranged on a bottom surface thereof.

2. A splint according to claim 1, wherein the reinforcement profile forms a support structure.

3. A splint according to claim 2, wherein the support structure is formed such that a bottom surface of the reinforcement profile lies in the same plane.

4. A splint according to claim 2, wherein the support structure is shaped to match a fixation fixture.

5. A splint according to claim 1, wherein the splint is comprised of a non-rigid material.

6. A radial artery compression system comprising a splint according to claim 1.

7. An arterial wrist support, comprising a splint according to claim 1.

8. A splint according to claim 5, wherein the non-rigid material is polystyrene.

9. A splint according to claim 5, wherein the non-rigid material is paper.

10. A splint according to claim 1, comprising at least two longitudinal reinforcement profiles having bottom surfaces which lie in the same plane.

11. A splint according to claim 1, further comprising a main body and wherein the main body and the at least one longitudinal reinforcement profile are formed from the same material.

12. A splint according to claim 1, wherein the reinforcement profile extends substantially along the full length of the splint.

13. A splint for a radial artery compression system, comprising:
an inner surface shaped to cradle at least one of a hand, wrist, and forearm of a patient; and
a support structure on an opposite side of the splint than the inner surface, the support structure including a substantially flat bottom surface.
